(19) United States
(12) Patent Application Publication
(10) Pub. No.: US 2006/0224239 A1
(43) Pub. Date: Oct. 5, 2006

(54) Breast implant

(76) Inventor: Leif K. Tiahrt, Santa Barbara, CA (US)

Correspondence Address: Michael G. Petit, P.O. Box 91929, Santa Barbara, CA, 93190-1929, US

(21) Appl. No.: 11/116,688

(22) Filed: Apr. 27, 2005

Related U.S. Application Data
(60) Provisional application No. 60/668,558, filed on Apr. 4, 2005.

(51) Int. Cl. A61F 2/12; A61F 2/02

(52) U.S. Cl. 623/8; 623/23.74

(57) ABSTRACT

A breast prosthesis has a flexible shell surrounding an interior compartment. An inner surface of the shell has a plurality of flexible protuberances or struts integral therewith that extend inwardly into the interior compartment. The opposing outer surface of the shell of the breast prosthesis is preferably textured to promote tissue ingrowth. A hole in the flexible shell allows the breast prosthesis to be made in an everted configuration, then everted to provide the prosthesis. In a preferred embodiment, the prosthesis does not contain a fluid filler material and is leak and rupture-proof. The breast prosthesis may be encased in a biocompatible elastomeric shell for implantation or be made from a suitable and inexpensive elastomer and worn as an explant. The prosthesis can be made in a variety of shapes for other types of soft tissue implants.

This application claims the benefit of U.S. Provisional Application Ser. No. 60/668,558, filed Apr. 4, 2005.

### BACKGROUND OF THE INVENTION

1. Field of the Invention

The present invention relates to a prosthesis for either implantation beneath the skin of a person or worn externally, and more particularly, to an implantable breast prosthesis.

2. Prior Art

Surgical reconstruction of human breasts as a result of injury or as a result of partial or total mastectomy has been performed for many years. Various surgical prostheses have been developed for implantation in the human breast as a part of breast reconstruction or augmentation. Two major problems with prostheses heretofore used is rupture and the lack of maintained projection, particularly in a manner such that the proper shape and location on the chest wall would be achieved and maintained, and the fact that their shape and consistency generally does not match the remaining breast. In an effort to overcome these problems a number of types of implants have been proposed.

U.S. Pat. No. 4,650,487 to Chaglassian discloses a high projection implant utilizing two shells with chambers therewithin in which the inner shell is secured to the rear wall of the outer shell and in which the fluid filling material contained in the inner chamber is of a higher density than that contained in the outer chamber. The prosthesis can, however, rupture; leaking filler material into the body.

U.S. Pat. No. 4,636,213 discloses a prosthesis which is provided with valves so that additional fluid can be introduced into the prosthesis by means of an injection needle to change the elevation of the prosthesis. Again, the prosthesis can rupture and release filler material into the body of the host.

Canadian Pat. No. 1,059,262 discloses an external breast prosthesis. Such external prostheses are simply attached over the chest area of the patient and may be removed at night if desired. The prosthesis shown in the '262 patent includes a silicone or other filler such as glycerin in which a bag of air is inserted to reduce its weight.

Silicone gel (and saline) filled implants are well known in the art and have received much attention due to their susceptability to leakage and rupture. Such implants have a shell comprised of a biocompatible elastomer such as silicone which is filled with a fluid such as saline or silicone gel, although other filler fluids can also be used. In accordance with the current art, the silicone shell is filled with a fluid filler material by means of injection into an interior chamber within the shell through a self-sealing valve in the shell. Such commercially available implants may further comprise a smaller inner shell having the same general shape (generally a flattened cushion or dome shape having a flattened rear side) having a height and diameter nearly as great as that of the outer shell to maintain a desired profile.

The prior art breast prostheses, generally described above, whether they comprise an inner shell (a “double lumen”) or not, more or less maintain the projection of the implant to varying degrees following implantation. Notwithstanding their advantages, such prior art fluid-filled implants may leak or rupture following implantation with possibly adverse effects on the host. There remains a need for an implantable breast prosthesis that is safe, leak and rupture-proof, easy to manufacture and which maintains a desired projection and profile after implantation.

### SUMMARY

The present invention is directed to a breast prosthesis which may or may not be fluid-filled, and a method for making the breast prosthesis that substantially obviates one or more of the limitations of the related art. To achieve these and other advantages and in accordance with the purpose of the invention, as embodied and broadly described herein, the invention is a soft tissue prosthesis, particularly a breast prosthesis, comprising a self-supporting flexible shell substantially surrounding an interior compartment. An inner surface of the shell has a plurality of flexible protruberances formed thereon that extend radially inwardly into the interior compartment. Preferably, at least a portion of an outer surface of the flexible shell has a rough texture operable for permitting tissue ingrowth into the outer surface of the shell when the breast prosthesis is implanted in a patient (host). The shell and the protuberances are preferably formed as a single body that is unitary in construction and made from a biocompatible elastomer. Alternatively, the protruberances may be made separately on a sheet and glued, vulcanized or otherwise adhered to the innner surface of the shell. The flexible shell of the breast prosthesis has a hole therein which permits the shell to be everted. If desired, the inner chamber of the prosthesis may be sealed by means of a patch adhered to said shell. If desired, the hole can remain open inasmuch as the protruberances act as flexible struts to maintain the profile and elevation of the prosthesis and no filler material is required in order to maintain the profile of the prosthesis. The patch, if used to seal the hole, may include a self-sealing injection port operable for introducing a fluid into the inner chamber within the flexible shell. Alternatively, the patch, if used, may be a sheet of biocompatible material lacking an injection port which serves to seal the inner chamber whether or not a fluid is contained in the inner chamber.

A first method for making the breast prosthesis includes the step of molding the shell and protuberances in a single mold with the protuberances projecting outwardly. The prosthesis is then removed from the mold and everted through a hole in the shell such that the plurality of protuberances project inwardly. After the shell is everted, the outer surface of the shell is preferably rough-textured.

In a second method, the shell is made by molding or by repeatedly dip casting an appropriately shaped mandrel into an elastomeric dispersion to form an elastomeric shell of an appropriate thickness. One or more elastomeric strips supporting a plurality of protruberances are glued, vulcanized or otherwise adhered to the surface of the shell. When the shell is everted (if molded) or removed from the mandrel and everted, the protruberances project inwardly from the (now) inner surface of the shell into the inner chamber of the shell. The plurality of protruberances may be the same length or include protruberances having a variety of lengths.

In both methods, the breast prosthesis is made from a biocompatible elastomer, preferably medical-grade silicone. The shape of the prosthesis can be varied to provide prostheses that conform to soft tissue implants other than breast implants such as biceps, calves and buttock by changing the shape of the mold (method 1) or the mold or mandrel (method 2). The relative lengths of the protruberances may also be selected so as to provide a prosthesis having the desired profile. Preferred lengths for the protruberances are such that the free ends 15 of the protruberances 13 are adjacent one another and disposed near the center of the inner chamber 16 of the prosthesis when the prosthesis 10 is in the implantable configuration as shown in FIGS. 1 and 3.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a schematic perspective illustration of a breast prosthesis in accordance with a preferred embodiment of the present invention.

FIG. 2 is a schematic perspective illustration of the breast prosthesis of FIG. 1 turned inside-out (everted).

FIG. 3 is a schematic cross-sectional illustration of the breast prosthesis of FIG. 1 taken along section line 3-3.

FIG. 4 is a schematic cross-sectional illustration of the everted breast prosthesis of FIG. 2 taken along section line 4-4.

FIG. 5 is a perspective view of a prosthesis in accordance with the present invention made by the method of adhering protruberance-bearing strips to the outer surface of the shell prior to everting the shell to form the prosthesis.

FIG. 6 is a perspective view of an embodiment of the present invention wherein an elastomeric body supporting a plurality of protruberances is inserted into an elastomeric shell to form an implantable prosthesis.

### DESCRIPTION OF THE PREFERRED EMBODIMENTS

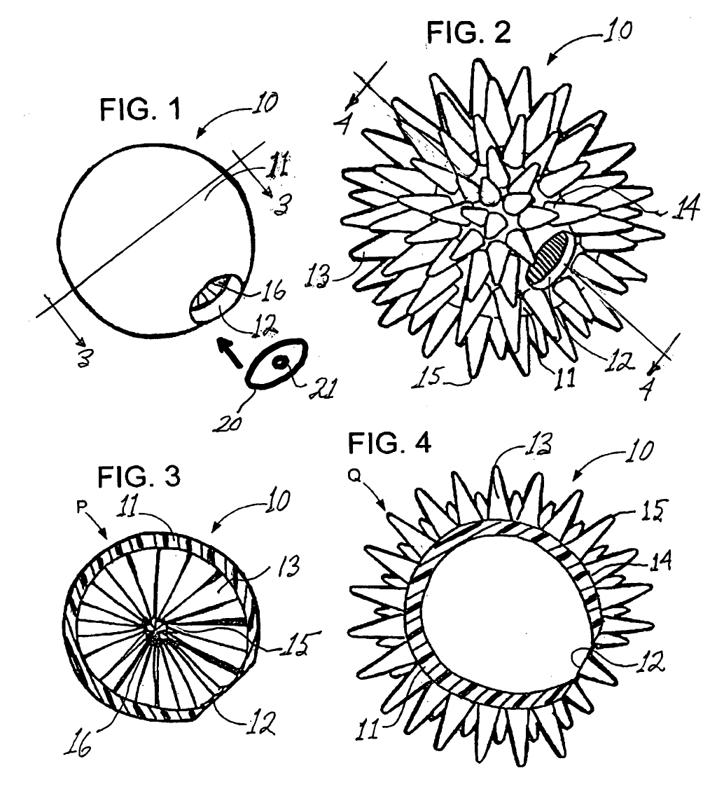

In FIG. 1 of the accompanying drawings there is schematically depicted an implantable breast prosthesis 10 in accordance with the present invention. Breast prosthesis 10 is preferably of unitary construction and is preferably formed in an everted configuration as shown in FIG. 2, by injecting a fluidic elastomer into a mold and curing the elastomer. The mold is separated, the (everted) breast prosthesis (FIG. 2) removed from the mold, then everted to provide the breast prosthesis 10 of FIG. 1. The inner surface of the shell 11 has a plurality of flexible elastomeric protruberances 13, preferably conical in shape, projecting radially inwardly from the surface of the shell and substantially filling the inner chamber of the prosthesis when the prosthesis is in the configuration shown in perspective view in FIG. 1 and cross-sectional view in FIG. 3. The term “flexible”, as used herein to describe a protruberance, means that the protruberance is elastically deformable.

Typically, the material employed to make both the shell 11 and the protruberances 13 is a moldable, medical-grade silicone elastomer. The shell and protruberances are preferably somewhat gelatinous in consistency, but sufficiently cohesive to maintain the shape and structural integrity of the prosthesis at body temperature, and to return to its desired shape following mechanically deformation thereof. The shell 11 of the breast prosthesis 10 is of an overall size and shape suitable for providing an esthetically pleasing appearance following implantation thereof within the body of a person (i.e., a “host” or “implant recipient”). The wall thickness of the shell 11 is preferably between 0.040 and 0.25 inches. The prosthsis 10, though preferably made from a biocompatible elastomer, may be made from a nonbiocompatible elastomer having a desired durometer, then coated with a durable, flexible, biocompatible elastomer as, for example, by dipping the prosthesis in a silicone dispersion to form a coating on the prosthesis, then curing the coat.

The shell 11 of the breast prosthesis, in both the everted (FIG. 2) and implantable configuration (FIG. 1), has a hole 12 therein. Within the shell (in the configuration of FIGS. 1 and 3) there is an interior compartment (also referred to herein as the, “inner chamber”) 16 into which the plurality of flexible protruberances 13 project. The protruberances 13 have a fixed end 14 adhered to, and preferably continuous with, the inner surface of the shell 11 and a free end 15 in opposition thereto. The length of the protruberances 13 are preferably such that the free ends 15 of the flexible protruberances 13 are adjacent to one another when the shell is everted in the “implantable configuration” shown in FIG. 1 and FIG. 3. The protruberances 13 serve as flexible struts that assist in maintaining the desired profile of the prosthesis 10. The outer surface of the shell 11 (the external side in FIG. 1) preferably has a roughened surface to encourage tissue ingrowth and discourage capsular contracture as is well known in the art. The breast prosthesis 10 can be everted by pushing at P (as shown in FIG. 3) to thereby force all of the protuberances 13 through the hole 12. The everted breast prosthesis has the configuration depicted in FIGS. 2 and 4. The everted breast prosthesis can be returned to the configuration of the implantable prosthesis shown in FIGS. 1 and 3 by pushing at Q (as shown in FIG. 4). It is preferable to implant the prosthesis 10 (FIG. 1) within a host without a filler material within the inner chamber 16. If it is so desired, a fluidic filler material such as silicone gel, saline, water or air, can be enclosed within the inner chamber 16 and sealed therein by affixing a patch 20 to the shell to seal the hole 12. The patch 20 may further include a self-sealing injection port 21 operable for passing a needle or cannula into the inner chamber to inject a fluid into, or remove a fluid from, the inner chamber.

The relative lengths of the flexible protruberances 13 may also be selected so as to provide a prosthesis having the desired profile. As stated above, the preferred lengths for the protruberances are such that the free ends 15 of the protruberances 13 are adjacent one another and disposed near the center of the inner chamber 16 of the prosthesis when the prosthesis 10 is in the implantable configuration as shown in FIGS. 1 and 3.

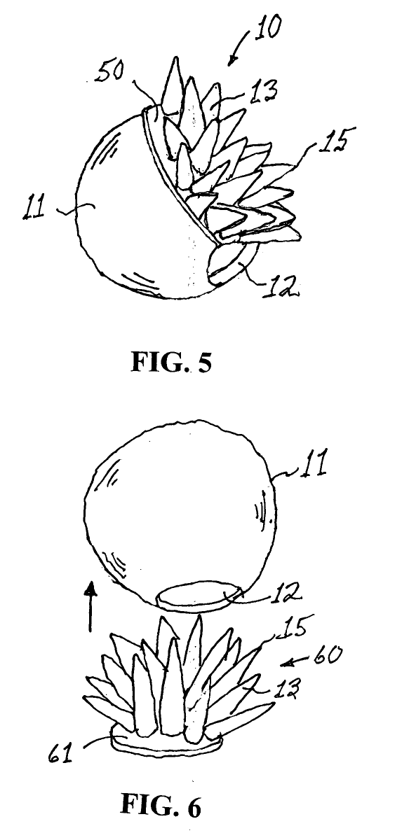

FIG. 5 is a perspective view of a prosthesis in accordance with the present invention made by yet another method wherein one or more protruberance-bearing strips 50 are adhered to the outer surface of the shell 11 prior to everting the shell to form the implantable configuration of the prosthesis. Again, the strips 50 and the protruberances 13 are preferably made from a biocompatible elastomer such as medical-grade silicone.

FIG. 6 is a perspective view of yet another embodiment of the present invention wherein an elastomeric body 60 supporting a plurality of protruberances 13 is inserted into an elastomeric shell 11 to form an implantable prosthesis. The body 60 is shown with conical protruberances 13 attached to a base portion 61 with the apex 15 of the protruberances 13 in opposition to the fixed end. The protruberances 13 may be such that the apex of the conical protruberances is affixed to the base portion. Further, the flexible protruberances 13 may be of other shapes such as a mushroom shape with the “stalk” of the mushroom attached to the base portion 61, or shaped like an umbrella with the handle of the umbrella affixed to the base portion 61. The protruberance 13 may be shaped like a loop wherein both ends of the loop are affixed to the shell, or it may comprise a spherical bead or hemisphere supported on a stem which is affixed to the base portion. As with other embodiments of the prosthesis of the present invention, the protruberances serve as flexible struts that support the shell 11. In this regard, it is preferable that the shell-contacting surface of the protruberances in accordance with this embodiment of the invention have a curvature that substantially conforms to the curvature of the inner surface of the shell at the point of contact therebetween.

While particular embodiments of the present invention have been illustrated and described, it would be obvious to those skilled in the art that various other changes and modifications can be made without departing from the spirit and scope of the invention. For example, the length and distribution of the flexible protruberances 13 on the inner surface of the shell 11 can be varied to change the physical characteristics of the prosthesis. Similarly, the roughness of the outer surface of the shell can be made by any one of a variety of methods known in the art. In addition, the implantable prosthesis can be made in a variety of shapes and sizes for augmenting or contouring soft tissue other than breast tissue. It is also contemplated that the prosthesis may be made using a suitable polymeric composition, which may or may not be biocompatible, then coated with a biocompatible elastomer, or the prosthesis 10 can be encased within a silicone shell of the type currently used in breast implants and the like and serve as a solid filler therefor. Further, the prosthesis 10 may contain a lubricant within the inner chamber to reduce friction between adjacent protruberances when the prosthesis is deformed. It is therefore intended to cover in the appended claims all such changes and modifications that are within the scope of this invention.

What I claim is:

1. A breast prosthesis comprising a flexible shell having an outer tissue-contacting surface and an inner surface substantially surrounding an interior compartment, said inner surface of said shell having a plurality of protruberances having a fixed end affixed to said inner surface and a free end in opposition to said fixed end that extends radially inwardly into said interior compartment.

2. The breast prosthesis of claim 1 wherein said shell of said breast prosthesis has a hole therein and wherein said protruberances are flexible.

3. The breast prosthesis of claim 1 wherein at least a portion of said tissue-contacting outer surface of said flexible shell has a rough texture operable for permitting tissue ingrowth into said tissue-contacting outer surface of said shell following implantation within a host.

4. The breast prosthesis of claim 2 wherein at least a portion of said tissue-contacting outer surface of said flexible shell has a rough texture operable for permitting tissue ingrowth into said tissue-contacting outer surface of said shell following implantation within a host.

5. The breast prosthesis of claim 1 wherein said protruberances are substantially conical in shape and wherein said free end of said protruberances is the apex of the conical protruberance.

6. The breast prosthesis of claim 1 wherein said breast prosthesis has unitary construction and is made from a biocompatible elastomer.

7. The breast prosthesis of claim 6 wherein said biocompatible elastomer is silicone.

8. The breast prosthesis of claim 1 formed by injection molding.

9. The breast prosthesis of claim 1 wherein said shell is formed by dipping a mandrel into a fluid elastomer.

10. A soft tissue prosthesis comprising a flexible shell having an outer tissue-contacting surface and an inner surface, said inner surface substantially surrounding an interior compartment, said inner surface of said shell having a plurality of flexible protruberances having a fixed end affixed to said inner surface and a free end in opposition to said fixed end that extends radially inwardly into said interior compartment.

11. The soft tissue prosthesis of claim 10 wherein said flexible shell has a hole therein.

12. The soft tissue prosthesis of claim 11 wherein said hole in said flexible shell is sealed by means of a patch adhered to said shell.

13. A prosthesis for implantation within the body of a person comprising an outer shell substantially enveloping an inner shell having an interior compartment, wherein an inner surface of said inner shell has a plurality of protruberances thereon that extend into said interior compartment.

14. The prosthesis of claim 13 wherein at least a portion of an outer surface of said outer shell has a rough texture operable for permitting tissue ingrowth into said outer surface of said shell.

15. The prosthesis of claim 13 wherein said inner shell has a hole therein.

16. The prosthesis of claim 15 wherein said outer shell has a hole therein that is sealed with a patch.

17. The prosthesis of claim 13 wherein said outer shell has an inner surface having a plurality of flexible protruberances thereon.

18. The prosthesis of claim 17 wherein an outer surface of said inner shell has a plurality of protruberances thereon.

19. The prosthesis of claim 13 wherein an outer surface of said inner shell has a plurality of protruberances thereon and wherein said inner surface of said inner shell is substantially smooth.
